(19) United States
(12) Patent Application Publication
(10) Pub. No.: US 2022/0033328 A1
(43) Pub. Date: Feb. 3, 2022

(54) METHOD OF PURIFYING MONOMER COMPOSITION AND METHOD OF PRODUCING POLYMER

(71) Applicant: ZEON CORPORATION, Chiyoda-ku, Tokyo (JP)

(72) Inventors: Yusuke YASU, Chiyoda-ku, Tokyo (JP); Kensaku FUJII, Chiyoda-ku, Tokyo (JP)

(73) Assignee: ZEON CORPORATION, Chiyoda-ku, Tokyo (JP)

(21) Appl. No.: 17/276,516

(22) PCT Filed: Jun. 25, 2019
(86) PCT No.: PCT/JP2019/025214

§ 371 Date: Mar. 16, 2021

(30) Foreign Application Priority Data
Sep. 26, 2018 (JP) 2018-180497
Sep. 26, 2018 (JP) 2018-180498

(51) Int. Cl. C07C 7/12; B01J 20/08; B01J 20/28; C08F 212/32; C08F 297/02; C08F 236/10

(52) U.S. Cl. CPC C07C 7/12; B01J 20/08; C08F 236/10; C08F 212/32; C08F 297/02; B01J 20/28061

(57) ABSTRACT

Provided is a method of purifying a monomer composition that contains a polycyclic aromatic vinyl compound including at least two monocycles selected from the group consisting of aromatic hydrocarbon monocycles and aromatic heteromonocycles. The purification method includes an impurity removal step of removing at least sulfur from the monomer composition.

### TECHNICAL FIELD

The present disclosure relates to a method of purifying a monomer composition and a method of producing a polymer.

### BACKGROUND

In recent years, materials derived from copolymers including monomer units that includes at least two aromatic hydrocarbon monocycles, such as vinylnaphthalene, and aliphatic conjugated diene monomer units have been attracting interest as excellent materials that can be used in various applications.

In one specific example, Patent Literature (PTL) 1 discloses a method of obtaining a hydrogenated block copolymer by using a metal catalyst to hydrogenate an A-B-A triblock copolymer that is obtained through a copolymerization reaction of a specific vinylnaphthalene and a specific diene, and also discloses an optical film formed of this hydrogenated block copolymer.

### CITATION LIST

### Patent Literature

PTL 1: JP2006-111650A

### SUMMARY

### Technical Problem

Studies carried out by the inventors have revealed that in production of a polymer using a monomer composition that contains a polycyclic aromatic vinyl compound including a specific vinylnaphthalene such as disclosed in PTL 1, impurities that may be contained in the polycyclic aromatic vinyl compound can impair progress of the polymerization reaction. However, PTL 1 does not in any way mention the influence that impurities contained in a polycyclic aromatic vinyl compound can have on a polymerization reaction and does not make any disclosure from a viewpoint of reducing the amount of such impurities.

Accordingly, one object of the present disclosure is to provide a method of purifying a monomer composition that can at least partially remove impurities capable of impairing a polymerization reaction that are contained in a monomer composition containing a polycyclic aromatic vinyl compound.

Another object of the present disclosure is to provide a method of producing a polymer using a composition that contains at least a polycyclic aromatic vinyl compound-containing monomer composition that has been purified.

### Solution to Problem

The inventors conducted diligent investigation to achieve the objects set forth above. The inventors reached new findings such as that in formation of a polymer using a monomer composition that contains a polycyclic aromatic vinyl compound, sulfur that may be contained as an impurity in the monomer composition containing the polycyclic aromatic vinyl compound acts to impair formation of the polymer. In this manner, the inventors completed the present disclosure.

Specifically, the present disclosure aims to advantageously solve the problem set forth above, and a presently disclosed method of purifying a monomer composition is a method of purifying a monomer composition containing a polycyclic aromatic vinyl compound including at least two monocycles selected from the group consisting of aromatic hydrocarbon monocycles and aromatic heteromonocycles, comprising an impurity removal step including removing at least sulfur from the monomer composition. By purifying a monomer composition containing a polycyclic aromatic vinyl compound to remove at least sulfur in this manner, it is possible to obtain a monomer composition that has a high polymerization conversion rate upon polymerization.

In the presently disclosed method of purifying a monomer composition, sulfur content in the monomer composition is preferably adjusted to 150 ppm or less based on mass of the polycyclic aromatic vinyl compound in the impurity removal step. By adjusting the sulfur content in the monomer composition to 150 ppm or less based on the mass of the polycyclic aromatic vinyl compound in the impurity removal step in this manner, it is possible to obtain a monomer composition having an even higher polymerization conversion rate upon polymerization. Note that the sulfur content can be measured by a method described in the EXAMPLES section.

In the presently disclosed method of purifying a monomer composition, sulfur content in a desulfurized monomer composition obtained through the impurity removal step is preferably 90 mass % or less of sulfur content in the monomer composition prior to purification. By removing at least 10 mass % of sulfur contained in the monomer composition prior to desulfurization in the impurity removal step in this manner, it is possible to obtain a monomer composition having an even higher polymerization conversion rate upon polymerization.

The presently disclosed method of purifying a monomer composition preferably further comprises removing halogen from the monomer composition in the impurity removal step. By removing halogen in purification of the specific monomer composition set forth above, it is possible to obtain a monomer composition having an even higher polymerization conversion rate upon polymerization.

In the presently disclosed method of purifying a monomer composition, halogen content in the monomer composition is preferably adjusted to 300 ppm or less based on mass of the polycyclic aromatic vinyl compound in the impurity removal step. By adjusting the halogen content in the monomer composition to 300 ppm or less based on the mass of the polycyclic aromatic vinyl compound in the impurity removal step, it is possible to obtain a monomer composition having an even higher polymerization conversion rate upon polymerization. Note that the halogen content can be measured by a method described in the EXAMPLES section.

In the presently disclosed method of purifying a monomer composition, halogen content in a dehalogenated monomer composition obtained through the impurity removal step is preferably 90 mass % or less of halogen content in the monomer composition prior to purification. By removing at least 10 mass % of halogen contained in the monomer composition prior to dehalogenation in the impurity removal step in this manner, it is possible to obtain a monomer composition having an even higher polymerization conversion rate upon polymerization.

In the presently disclosed method of purifying a monomer composition, the polycyclic aromatic vinyl compound may include vinylnaphthalene. A monomer composition containing vinylnaphthalene can suitably be used in various applications.

In the presently disclosed method of purifying a monomer composition, sulfur is preferably removed from the monomer composition by adding an adsorbent to the monomer composition in the impurity removal step. By adding an adsorbent to a monomer composition containing a polycyclic aromatic vinyl compound so as to remove sulfur in this manner, it is possible to obtain a monomer composition having a high polymerization conversion rate upon polymerization.

In the presently disclosed method of purifying a monomer composition, the adsorbent preferably includes more than 50 mass % of Al₂O₃. When an adsorbent that satisfies the chemical composition set forth above is used in this manner, sulfur can be more effectively removed from the monomer composition. Note that the chemical composition of an adsorbent can be analyzed by a method described in the EXAMPLES section.

In the presently disclosed method of purifying a monomer composition, the adsorbent preferably has a BET specific surface area of 100 m²/g or more. When the BET specific surface area of the adsorbent is 100 m²/g or more in this manner, sulfur can be more effectively removed from the monomer composition. Note that the BET specific surface area of an adsorbent can be measured by a method described in the EXAMPLES section.

In the presently disclosed method of purifying a monomer composition, the adsorbent is preferably added in a proportion of at least 0.05 times mass of the polycyclic aromatic vinyl compound in the impurity removal step. By setting the additive amount of the adsorbent as at least 0.05 times the mass of the polycyclic aromatic vinyl compound, sulfur can be more effectively removed from the monomer composition.

Moreover, the present disclosure aims to advantageously solve the problem set forth above, and a presently disclosed method of producing a polymer comprises a polymerization step of performing anionic polymerization of a composition (I) containing a purified monomer composition obtained by any one of the methods of purifying a monomer composition set forth above to obtain a polymer. By performing anionic polymerization of the composition (I), a polymer can be formed with a high polymerization conversion rate.

In the presently disclosed method of producing a polymer, the polycyclic aromatic vinyl compound is preferably copolymerized with an aliphatic conjugated diene compound in the polymerization step. By copolymerizing the polycyclic aromatic vinyl compound with an aliphatic conjugated diene compound in the polymerization step, it is possible to obtain a copolymer that can suitably be used in various applications and that, in particular, can suitably be used for forming an optical component such as an optical film.

The presently disclosed method of producing a polymer may further comprise (1) block copolymerizing the polycyclic aromatic vinyl compound and the aliphatic conjugated diene compound to obtain a block copolymer or (2) randomly copolymerizing the polycyclic aromatic vinyl compound and the aliphatic conjugated diene compound to obtain a random copolymer in the polymerization step. Depending on the polymerization structure of a block copolymer, random copolymer, or the like, it is possible to impart desired attributes to a polymer, and thus it is possible to provide polymers that can suitably be adopted in various applications.

### Advantageous Effect

According to the present disclosure, it is possible to provide a method of purifying a monomer composition that can at least partially remove impurities capable of impairing a polymerization reaction that are contained in a monomer composition containing a polycyclic aromatic vinyl compound.

Moreover, according to the present disclosure, it is possible to provide a method of producing a polymer using a composition that contains at least a polycyclic aromatic vinyl compound-containing monomer composition that has been purified.

### DETAILED DESCRIPTION

The following provides a detailed description of embodiments of the present disclosure.

A purified monomer composition obtained by the presently disclosed method of purifying a monomer composition can suitably be used in the presently disclosed method of producing a polymer.

(Method of Purifying Monomer Composition)

The presently disclosed method of purifying a monomer composition is a method of purifying a monomer composition that contains a polycyclic aromatic vinyl compound including at least two monocycles selected from the group consisting of aromatic hydrocarbon monocycles and aromatic heteromonocycles. A feature of the purification method is that it includes an impurity removal step of removing at least sulfur from the monomer composition. In the presently disclosed method of purifying a monomer composition, a monomer composition that has a high polymerization conversion rate upon polymerization is obtained as a purified monomer composition by removing sulfur contained as an impurity in the monomer composition that is a subject of purification. Note that when a given monomer composition is said to have a high polymerization conversion rate upon polymerization, this means that a high yield is achieved when the monomer composition is used to obtain a polymer. Therefore, a monomer composition that has a high polymerization conversion rate upon polymerization is advantageous from a viewpoint of efficiently producing a polymer. In addition, the impurity removal step in the presently disclosed method of purifying a monomer composition preferably includes removing halogen. The following describes the monomer composition that is the purification subject, various steps that can be included in the presently disclosed method of purifying a monomer composition, and so forth.

<Monomer Composition that is Purification Subject>

The monomer composition that is the purification subject in the presently disclosed method of purifying a monomer composition contains a specific polycyclic aromatic vinyl compound and impurities.

<<Polycyclic Aromatic Vinyl Compound>>

The polycyclic aromatic vinyl compound is a compound that includes at least two monocycles selected from the group consisting of aromatic hydrocarbon monocycles and aromatic heteromonocycles. Note that the two or more monocycles that are present in the polycyclic aromatic vinyl compound may be independent of one another or may be fused to form a fused ring, but it is preferable that two or more monocycles that are present are present in a fused state. Moreover, the monomer composition that is the purification subject may contain one polycyclic aromatic vinyl compound or a plurality of polycyclic aromatic vinyl compounds.

Examples of aromatic hydrocarbon monocycles include a benzene ring and a substituted benzene ring. Examples of possible substituents include alkyl groups such as a methyl group, an ethyl group, a propyl group, and a t-butyl group; and halogen groups such as a fluoro group, a chloro group, and a bromo group.

Examples of aromatic heteromonocycles include an oxadiazole ring, an oxazole ring, an oxazolopyrazine ring, an oxazolopyridine ring, an oxazolopyridazyl ring, an oxazolopyrimidine ring, a thiadiazole ring, a thiazole ring, a triazine ring, a pyranone ring, a pyran ring, a pyridine ring, a pyridazine ring, a pyrimidine ring, and a pyrrole ring.

The polycyclic aromatic vinyl compound may be 1-vinylnaphthalene, 2-vinylnaphthalene, vinylanthracene, 1,1-diphenylethylene, or the like. Of these examples, vinylnaphthalene such as 1-vinylnaphthalene and 2-vinylnaphthalene is preferable as the polycyclic aromatic vinyl compound. A monomer composition that contains vinylnaphthalene can suitably be used in various applications.

<<Impurities>>

Examples of impurities that may be contained in the monomer composition that is the purification subject include sulfur. The sulfur contained as an impurity is not specifically limited and can be contained as free sulfur and sulfur substances such as various sulfur-containing compounds. Examples of impurities other than sulfur include halogen such as fluorine, chlorine, bromine, and iodine, and water. The form in which halogen is contained as an impurity is not specifically limited, and halogen can be contained as various halogen-containing compounds.

Studies carried out by the inventors have revealed that among the impurities described above, sulfur that can be contained in various forms contributes significantly to an effect of impairing the polymerization conversion rate during polymerization of the monomer composition. Accordingly, the removal of at least sulfur from the monomer composition in the impurity removal step is an essential requirement in the presently disclosed method of purifying a monomer composition.

<Impurity Removal Step>

In the impurity removal step, at least sulfur is removed from the monomer composition. The method by which sulfur is removed is not specifically limited, and a method using an adsorbent, a sublimation method, or the like can be adopted. Of these methods, a method using an adsorbent is preferable from a viewpoint of efficiency of sulfur removal. Specifically, in a method using an adsorbent, sulfur contained in the monomer composition that is the purification subject can be removed by bringing an adsorbent into contact with the monomer composition.

<<Adsorbent>>

The adsorbent can be any substance so long as it can adsorb at least free sulfur or a sulfur compound. The adsorbent is preferably an inorganic oxide having a chemical composition in which the proportion constituted by Al₂O₃ is more than 50 mass %. The proportion constituted by Al₂O₃ in the chemical composition of an inorganic oxide serving as the adsorbent is more preferably 60 mass % or more, more preferably 75 mass % or more, and even more preferably 95 mass % or more. Sulfur can be more effectively removed from the monomer composition when Al₂O₃ has a high proportional content in the chemical composition of an inorganic oxide serving as the adsorbent. Note that the chemical composition of an inorganic oxide serving as the adsorbent may be composed of substantially only Al₂O₃ (i.e., high-purity alumina may be used as the adsorbent). Moreover, an inorganic oxide serving as the adsorbent preferably contains Fe₂O₃. In a case in which an inorganic oxide serving as the adsorbent contains Fe₂O₃, the proportional content of Fe₂O₃ can be not less than 0.01 mass % and not more than 0.05 mass %.

The BET specific surface area of the adsorbent is preferably 100 m²/g or more, and more preferably 150 m²/g or more. Sulfur can be more effectively removed from the monomer composition when the BET specific surface area of the adsorbent is not less than any of the lower limits set forth above. Note that the BET specific surface area of the adsorbent can normally be 400 m²/g or less.

The number-average particle diameter of the adsorbent is preferably 10 mm or less. When the number-average particle diameter is 10 mm or less, contact frequency of the adsorbent and the monomer composition can be sufficiently increased, and sulfur removal efficiency can be increased. Note that the shape of the adsorbent is preferably a spherical shape. This is because contact frequency of the adsorbent and the monomer composition can be sufficiently increased when the adsorbent has a spherical shape. Moreover, the number-average particle diameter of the adsorbent can normally be 1 mm or more. Note that the number-average particle diameter of an adsorbent can be calculated by a method described in the EXAMPLES section as a number-average value of measured values for the diameters of 10 particles of the adsorbent.

The packing density of the adsorbent is preferably not less than 0.70 kg/L and not more than 0.90 kg/L. This is because sulfur removal efficiency can be more sufficiently increased when the packing density of the adsorbent is within the range set forth above. In the case of adsorbents having equivalent specific surface areas, an adsorbent having a larger packing density tends to provide better sulfur removal efficiency. Note that the packing density of an adsorbent can be measured by a standard method.

Examples of adsorbents that can satisfy preferred properties such as set forth above include, but are not specifically limited to, the KH series and the NK series of activated alumina produced by Sumitomo Chemical Co., Ltd.

In addition of the adsorbent to the monomer composition in the impurity removal step, the adsorbent is preferably added such that the added mass of the adsorbent is at least 0.05 times, more preferably at least 0.10 times, and even more preferably at least 0.15 times the mass of the polycyclic aromatic vinyl compound, and is preferably added such that the added mass of the adsorbent is at most 2.0 times the mass of the polycyclic aromatic vinyl compound. When the additive amount of the adsorbent as a proportion (times) relative to the mass of the polycyclic aromatic vinyl compound is not less than any of the lower limits set forth above, sulfur removal efficiency can be further increased. Moreover, when the additive amount of the adsorbent as a proportion (times) relative to the mass of the polycyclic aromatic vinyl compound is not more than the upper limit set forth above, it is possible to inhibit excessive reduction of the amount of the monomer composition that can be introduced into a vessel in which the impurity removal step is performed as a result of the volume occupied by the adsorbent becoming excessively large.

The impurity removal step can be performed under a temperature condition of not lower than −80° C. and not higher than 70° C. for a treatment time (period) of not less than 0.5 hours and not more than 30 days, for example.

In the impurity removal step, the sulfur content in the monomer composition is preferably adjusted to 150 ppm or less, and more preferably 100 ppm or less based on the mass of the polycyclic aromatic vinyl compound. By adjusting the sulfur content in the monomer composition to not more than any of the upper limits set forth above in the impurity removal step, it is possible to obtain a monomer composition having an even higher polymerization conversion rate upon polymerization. Note that sulfur may be completely removed in the impurity removal step (i.e., the impurity removal step may be performed under conditions such that the sulfur content in a desulfurized monomer composition that has undergone the impurity removal step is 0 ppm). However, the sulfur content in a desulfurized monomer composition that has undergone the impurity removal step may be 30 ppm or more from viewpoints such as not excessively increasing the time required for the impurity removal step and efficiently increasing the polymerization conversion rate upon polymerization.

In the impurity removal step, the sulfur content in a desulfurized monomer composition obtained through the impurity removal step is preferably adjusted to 90 mass % or less of the sulfur content in the monomer composition prior to purification, and more preferably 70 mass % or less of the sulfur content in the monomer composition prior to purification. By adjusting the ratio of the sulfur content in a desulfurized monomer composition that has undergone the impurity removal step relative to the sulfur content in the monomer composition prior to purification (hereinafter, also referred to simply as the “ratio relative to pre-purification sulfur content”) to not more than any of the upper limits set forth above, it is possible to obtain a monomer composition having an even higher polymerization conversion rate upon polymerization. Although the reason for this is not clear, it is presumed to be due to sulfur substances that have a large inhibitive effect on a polymerization reaction being efficiently removed in a situation in which treatment is performed to the extent that the ratio relative to pre-purification sulfur content is not more than any of the upper limits set forth above.

It is preferable that removal of halogen is also included in the impurity removal step of the presently disclosed method of purifying a monomer composition. By removing halogen in the impurity removal step, it is possible to obtain a monomer composition having an even higher polymerization conversion rate upon polymerization. Of halogens, it is preferable that bromine is removed in the impurity removal step, and, more specifically, it is preferable that the bromine content in the monomer composition is adjusted to within a preferred range such as subsequently described in the impurity removal step. Note that sulfur removal and halogen removal may be performed in any order in the impurity removal step. More specifically, one of sulfur removal and halogen removal may be performed first and then the other thereof may be performed afterwards, or both sulfur removal and halogen removal may be performed at the same time. The method by which halogen is removed is not specifically limited, and a method using an adsorbent, a sublimation method, a distillation method, or the like can be adopted.

For example, when an adsorbent is used to remove sulfur in the impurity removal step as previously described, there are instances in which the adsorbent adsorbs not only sulfur, but also halogen. In such a situation, an operation performed with the aim of removing sulfur in the impurity removal step also causes adsorption of halogen to the adsorbent, and thus results in sulfur removal and halogen removal being performed substantially at the same time. In this manner, sulfur removal and halogen removal may be performed substantially at the same time through a single operation in the impurity removal step.

In removal of halogen in the impurity removal step, the halogen content (preferably bromine content) in the monomer composition is preferably adjusted to 300 ppm or less, more preferably 250 ppm or less, and even more preferably 160 ppm or less based on the mass of the polycyclic aromatic vinyl compound. By adjusting the halogen content in the monomer composition to not more than any of the upper limits set forth above in the impurity removal step, it is possible to obtain a monomer composition having an even higher polymerization conversion rate upon polymerization. Note that halogen may be completely removed in the impurity removal step (i.e., halogen removal may be performed under conditions such that the halogen content in a dehalogenated monomer composition obtained through the impurity removal step is 0 ppm). However, the halogen content in a dehalogenated monomer composition obtained through the impurity removal step may be 50 ppm or more from a viewpoint of the time and workload required in the impurity removal step and a viewpoint of the polymerization conversion rate upon polymerization.

In the impurity removal step, the halogen content (preferably bromine content) in a dehalogenated monomer composition obtained through the impurity removal step is preferably adjusted to 90 mass % or less, more preferably 70 mass % or less, and even more preferably 55 mass % or less of the halogen content (preferably bromine content) in the monomer composition prior to purification. By adjusting the ratio of the halogen content in a dehalogenated monomer composition that has undergone the impurity removal step relative to the halogen content in the monomer composition prior to purification (hereinafter, also referred to simply as the “ratio relative to pre-purification halogen content”) to not more than any of the upper limits set forth above, it is possible to obtain a monomer composition having an even higher polymerization conversion rate upon polymerization.

<Other Pretreatment Steps>

A pretreatment step of removing water or the like contained in the monomer composition can optionally be performed. The method by which water contained in the monomer composition is removed is not specifically limited and may, for example, be a method in which the monomer composition is brought into contact with a desiccant formed of a zeolite, such as a molecular sieve. The pretreatment step can be performed at any timing without any specific limitations but is preferably performed at the same time as the impurity removal step set forth above from a viewpoint of increasing production efficiency.

(Method of Producing Polymer)

A feature of the presently disclosed method of producing a polymer is that it includes a polymerization step of performing anionic polymerization of a composition (I) containing a purified monomer composition obtained by the presently disclosed method of purifying a monomer composition set forth above to obtain a polymer. By performing anionic polymerization of the composition (I), a polymer can be formed with a high polymerization conversion rate. Although the reason for this is not clear, it is presumed that sulfur removed in the impurity removal step acts to impair anionic polymerization, in particular, among various polymerization methods, and thus the polymerization conversion rate by anionic polymerization can be significantly increased by using a purified monomer composition that has undergone the impurity removal step set forth above.

<Polymerization Step>

In the polymerization step, anionic polymerization of the composition (I) is performed to obtain a polymer. The composition (I) is required to contain the previously described polycyclic aromatic vinyl compound including at least two monocycles selected from the group consisting of aromatic hydrocarbon monocycles and aromatic heteromonocycles. By performing anionic polymerization using an anionic polymerization catalyst in the polymerization step, it is possible to obtain a homopolymer composed of only monomer units derived from the polycyclic aromatic vinyl compound or a copolymer including monomer units derived from the polycyclic aromatic vinyl compound and monomer units derived from one or more other compounds. Note that the anionic polymerization can be performed in an organic solvent under an inert gas atmosphere of nitrogen gas or the like without any specific limitations.

The polycyclic aromatic vinyl compound can be copolymerized with an aliphatic conjugated diene compound in the polymerization step. The aliphatic conjugated diene compound may be a chain conjugated diene compound such as 1,3-butadiene or 2-methyl-1,3-butadiene (isoprene), for example, with 2-methyl-1,3-butadiene (isoprene) and 1,3-butadiene being particularly preferable because they can impart flexibility to a copolymer composition after polymerization. One of these aliphatic conjugated diene compounds may be used individually, or two or more of these aliphatic conjugated diene compounds may be used as a mixture.

The composition (I) may contain other compounds besides the compounds described above. Examples of such other compounds include monocyclic aromatic vinyl compounds and unsaturated carboxylic acid esters. A monocyclic aromatic vinyl compound is a monomer that includes one of the previously described aromatic hydrocarbon monocycles. More specifically, styrene, α-methylstyrene, 2-methylstyrene, 3-methylstyrene, 4-methylstyrene, or the like can be used as a monocyclic aromatic vinyl compound. Moreover, specific examples of unsaturated carboxylic acid esters that may be used include methyl acrylate and methyl methacrylate. One of these other compounds may be used individually, or two or more of these other compounds may be used as a mixture.

The anionic polymerization catalyst used in the polymerization step may, for example, be an alkyllithium compound in which the alkyl group has a carbon number of 1 to 10. When sulfur is present in the composition (I) in a case in which an alkyllithium having an alkyl group carbon number of 1 to 10 is used as the anionic polymerization catalyst, it is presumed that anionic polymerization is impaired by the alkyllithium acting as a base, for example. Therefore, in a case in which an alkyllithium having an alkyl group carbon number of 1 to 10 is used as the anionic polymerization catalyst, impairment of the anionic polymerization reaction can be effectively inhibited through sulfur contained in a monomer composition being at least partially removed through the presently disclosed purification method.

The alkyllithium compound may be methyllithium, ethyllithium, pentyllithium, n-butyllithium, sec-butyllithium, t-butyllithium, or the like, for example. Of these alkyllithium compounds, n-butyllithium is preferably used as the anionic polymerization catalyst from a viewpoint of causing the polymerization reaction to progress efficiently. The amount of the anionic polymerization catalyst that is used can be adjusted as appropriate depending on the target copolymer molecular weight and may, for example, be within a range such that the number of molar equivalents of monomer in the composition (I) relative to the anionic polymerization catalyst is preferably 50 equivalents or more, and more preferably 100 equivalents or more, and is preferably 3,000 equivalents or less, more preferably 2,000 equivalents or less, and even more preferably 1,400 equivalents or less. Note that a known co-catalyst such as dibutyl ether may optionally be used in combination with the anionic polymerization catalyst.

Examples of organic solvents that can be used in the polymerization step include, but are not specifically limited to, aliphatic hydrocarbons such as pentane, hexane, and heptane; alicyclic hydrocarbons such as cyclopentane, cyclohexane, methylcyclohexane, dimethylcyclohexane, trimethylcyclohexane, ethylcyclohexane, diethylcyclohexane, decahydronaphthalene, bicycloheptane, tricyclodecane, hexahydroindene, and cyclooctane; aromatic hydrocarbons such as benzene, toluene, and xylene; halogenated aliphatic hydrocarbons such as dichloromethane, chloroform, and 1,2-dichloroethane; halogenated aromatic hydrocarbons such as chlorobenzene and dichlorobenzene; nitrogen-containing hydrocarbon solvents such as nitromethane, nitrobenzene, and acetonitrile; and mixed solvents of any of preceding organic solvents. Of these examples, toluene is preferably used as an organic solvent from a viewpoint of causing the polymerization reaction to progress efficiently. Note that one of these organic solvents may be used individually, or two or more of these organic solvents may be used in combination in a freely selected ratio. The amount of the organic solvent can, for example, be not less than 20 parts by mass and not more than 20,000 parts by mass when the total mass of monomer in the composition (I) is taken to be 100 parts by mass.

The method by which the polycyclic aromatic vinyl compound and the aliphatic conjugated diene compound are copolymerized may be a method in which the polycyclic aromatic vinyl compound and the aliphatic conjugated diene compound are randomly copolymerized or a method in which the polycyclic aromatic vinyl compound and the aliphatic conjugated diene compound are block copolymerized.

Any commonly known method of producing a random copolymer can be adopted without any specific limitations as the method by which the polycyclic aromatic vinyl compound and the aliphatic conjugated diene compound are randomly polymerized. In a case in which the polycyclic aromatic vinyl compound and the aliphatic conjugated diene compound are randomly polymerized, a random polymer can be formed with a high polymerization conversion rate as a result of the polycyclic aromatic vinyl compound being a purified monomer composition that has been obtained by the presently disclosed purification method. The proportional content of the polycyclic aromatic vinyl compound in the composition (I) in a case in which the polycyclic aromatic vinyl compound and the aliphatic conjugated diene compound are randomly polymerized can, for example, be not less than 5 mass % and not more than 99 mass % when all monomer in the composition (I) is taken to be 100 mass %. Particularly in a case in which the proportional content of the polycyclic aromatic vinyl compound in the composition (I) is not less than the lower limit set forth above, the polymerization conversion rate can be significantly increased as a result of a purified monomer composition that has been obtained by the presently disclosed purification method being used.

Moreover, any commonly known method of producing a block copolymer can be adopted without any specific limitations as the method by which the polycyclic aromatic vinyl compound and the aliphatic conjugated diene compound are block copolymerized. For example, in a case in which an [A]-[B]-[A] triblock copolymer including a polymer block [A] that includes polycyclic aromatic vinyl compound-derived structural units and has aromatic vinyl compound-derived structural units as a main component (hereinafter, also referred to simply as a “specific polymer block [A]”) and a polymer block [B] that has aliphatic conjugated diene compound-derived structural units as a main component (hereinafter, also referred to simply as a “specific polymer block [B]”) is to be produced, the method may be:

(i) a method including a first polymerization step of polymerizing a monomer mixture (a1) containing the polycyclic aromatic vinyl compound to form a specific polymer block [A], a second polymerization step of polymerizing a monomer mixture (b1) containing the aliphatic conjugated diene compound to form a specific polymer block [B], and a third polymerization step of polymerizing a monomer mixture (a2) containing the polycyclic aromatic vinyl compound to form a specific polymer block [A]; or

(ii) a method including a first polymerization step of polymerizing a monomer mixture (a1) containing the polycyclic aromatic vinyl compound to form a specific polymer block [A], a second step of polymerizing a monomer mixture (b1) containing the aliphatic conjugated diene compound to form a polymer block [B′] having aliphatic conjugated diene compound-derived structural units as a main component, and a step of coupling ends of polymer blocks [B′] to each other using a coupling agent.

Note that commonly known coupling agents can be used without any specific limitations as the coupling agent used in method (ii). Moreover, the amount of the coupling agent that is used can be adjusted as appropriate depending on the target block copolymer molecular weight. Also note that when a given polymer block is said to have given structural units “as a main component” in the present specification, this means that when all units forming the polymer block are taken to be 100 mass %, the proportion constituted by the given structural units is more than 50 mass %, and is preferably 60 mass % or more, more preferably 70 mass % or more, and even more preferably 80 mass % or more. Furthermore, in a case in which a given polymer block has given structural units “as a main component”, all units of the polymer block may be the given structural units. In other words, the given polymer block may be a block composed of only the structural units that are the main component thereof. Moreover, the mass ratio of compounds in a monomer mixture used to form such a polymer block is in accordance with the proportion constituted by each type of structural unit in the target polymer block.

Also note that the monomer mixture (a1) containing the polycyclic aromatic vinyl compound may contain a monocyclic aromatic vinyl compound. When the amount of all monomers contained in the monomer mixture (a1) is taken to be 100 mass %, the content of aromatic vinyl compounds, inclusive of the polycyclic aromatic vinyl compound and the monocyclic aromatic vinyl compound, is preferably more than 50 mass %, more preferably 60 mass % or more, even more preferably 70 mass % or more, and particularly preferably 80 mass % or more. Note that all monomers contained in the monomer mixture (a1) may be aromatic vinyl compounds. Moreover, the ratio of the polycyclic aromatic vinyl compound and the monocyclic aromatic vinyl compound in the monomer mixture (a1), by mass, can be within a range of polycyclic aromatic vinyl compound:monocyclic aromatic vinyl compound=5:95 to 100:0. The proportion constituted by each type of monomer unit in the polymer block [A] obtained through polymerization of the monomer mixture (a1) is a proportion in accordance with the content and ratio described above.

The polymerization temperature is not specifically limited and can be set as not lower than 20° C. and not higher than 150° C., and preferably not lower than 25° C. and not higher than 120° C., for example. This is because the polymerization catalyst can be caused to sufficiently function when the polymerization temperature is not lower than any of the lower limits set forth above. Moreover, decomposition of the polymerization catalyst can be inhibited when the polymerization temperature is not higher than any of the upper limits set forth above.

The polymerization time is not specifically limited and can be set as not less than 1 hour and not more than 10 hours, for example. This is because the polymerization reaction can be caused to sufficiently progress when the polymerization time is not less than the lower limit set forth above. Moreover, the time required for production of the copolymer can be reduced when the polymerization time is not more than the upper limit set forth above.

No specific limitations are placed on the method by which the resultant copolymer is collected once the polymerization step has ended. For example, the copolymer can be collected, as obtained, in the form of a polymerization solution. Note that the reaction mixture obtained through the polymerization step normally contains the copolymer and the organic solvent.

The polymer obtained through the polymerization step using the purified monomer composition includes structural units derived from the polycyclic aromatic vinyl compound and can optionally include structural units derived from an aliphatic conjugated diene compound and structural units derived from other compounds. More specifically, the polymer obtained through the polymerization step can be a homopolymer composed of only structural units derived from the polycyclic aromatic vinyl compound or can be a random copolymer or a block copolymer that can also include structural units derived from an aliphatic conjugated diene compound and structural units derived from any other compounds. The polymer has a narrow molecular weight distribution compared to a polymer produced through polymerization by a conventional method without using a purified monomer composition. Accordingly, the presently disclosed method of producing a polymer makes it possible to obtain a polymer having a comparatively uniform molecular weight. Note that a “molecular weight distribution” can be measured by a method described in the EXAMPLES section.

In a case in which a block copolymer present in a reaction mixture obtained by performing block copolymerization in the polymerization step is a triblock copolymer having an [A]-[B]-[A] triblock structure, the purity thereof is preferably 45% or more. Such a block copolymer can suitably be used in production of a material for forming an optical component. Note that “triblock copolymer purity” can be calculated by a method described in the EXAMPLES section as a ratio of the mass of triblock copolymer relative to the total mass of formed block copolymer.

The triblock copolymer purity is preferably 70% or more, and more preferably 90% or more. When the triblock copolymer purity is not less than any of the lower limits set forth above, even better optical performance such as three-dimensional retardation can be displayed in a situation in which a material containing the copolymer is shaped into a film shape.

Moreover, in a case in which a block copolymer present in a reaction mixture obtained by performing block copolymerization in the polymerization step is a triblock copolymer having an [A]-[B]-[A] triblock structure, the molecular weight distribution is preferably 1.50 or less. Such a block copolymer can suitably be used in production of a material for forming an optical component.

The molecular weight distribution of the triblock copolymer is preferably 2.0 or less, and more preferably 1.50 or less. When the molecular weight distribution of the obtained triblock copolymer is not more than any of the upper limits set forth above, even better optical performance such as three-dimensional retardation can be displayed in a situation in which a material containing the copolymer is shaped into a film shape.

### EXAMPLES

The following provides a more specific description of the present disclosure based on examples. However, the present disclosure is not limited to these examples. In the following description, “%” and “parts” used in expressing quantities are by mass, unless otherwise specified. Measurement methods of various physical properties are described below.

<Chemical Composition of Adsorbent>

An adsorbent was ground in a boron nitride mortar, and then the chemical composition thereof was determined by inductively coupled plasma (ICP) optical emission spectroscopy. The results are shown in the tables or are described herein.

### <BET Specific Surface Area of Adsorbent>

The BET specific surface area of an adsorbent was measured by the BET method based on JIS Z 8830:2013. The results are shown in Table 3.

### <Particle Diameter of Adsorbent>

The diameters of 10 adsorbent particles having a spherical shape were measured using a caliper, and a number-average value was calculated. The results are shown in Table 3.

<Number-Average Molecular Weight (Mn), Weight-Average Molecular Weight (Mw), Peak Top Molecular Weight (Mp), and Molecular Weight Distribution (Mw/Mn)>

Gel permeation chromatography (GPC) was used to measure the number-average molecular weight (Mn), the weight-average molecular weight (Mw), and the peak top molecular weight (Mp) and to calculate the molecular weight distribution (Mw/Mn) for each type of polymer obtained in the examples and comparative examples.

In this measurement, an HLC-8320 (produced by Tosoh Corporation) was used as a measurement instrument. Two TSKgel α-M columns (produced by Tosoh Corporation) connected in series were used as a column. A differential refractometer RI-8320 (produced by Tosoh Corporation) was used as a detector. Moreover, the number-average molecular weight (Mn), the weight-average molecular weight (Mw), and the peak top molecular weight (Mp) were determined as standard polystyrene-equivalent values for each type of polymer using tetrahydrofuran as an eluent solvent. The molecular weight distribution (Mw/Mn) was calculated from the determined values.

<Conversion Rates>

The conversion rate of 2-vinylnaphthalene and the conversion rate of isoprene were calculated through ¹H-NMR measurement with deuterated chloroform as a solvent. An evaluation was made by the following standard in some of the examples.

A: 90% or more

B: Not less than 80% and less than 90%

C: Less than 80%

<Measurement of Sulfur Content and Bromine Content>

A purified monomer composition obtained in each example or comparative example was distilled under reduced pressure to remove solvent and obtain a sample. Approximately 0.02 g of the sample was weighed onto a magnetic board, the sample was combusted by an automatic combustion device (produced by Yanaco), and then the sulfur content and the bromine content were quantified by ion chromatography (ICS-1500 produced by Dionex Corporation). The sulfur content and the bromine content were quantified as the amount (μg) of sulfur and the amount (μg) of bromine contained per 1 g of mass of a polycyclic aromatic vinyl compound (i.e., as an amount (ppm) based on the mass of the polycyclic aromatic vinyl compound).

Also note that a ratio relative to pre-purification sulfur content and a ratio relative to pre-purification bromine content can be obtained by dividing the sulfur content (ppm) and the bromine content (ppm) in the purified monomer composition, quantified as described above, by the sulfur content (ppm) and the bromine content (ppm) in the monomer composition prior to purification, and converting the resulting values to percentages.

<Triblock Copolymer Purity>

In each example or comparative example in which a triblock copolymer was produced, the triblock copolymer purity was calculated based on the following equation.

Triblock copolymer purity (%)=[Mass of isolated triblock copolymer (g)/Mass of all polymer contained in reaction mixture (g)]×100

Note that the mass of each polymer contained in a reaction mixture was calculated based on an area ratio according to gel permeation chromatography (GPC).

The following demonstrates that it was confirmed through Examples 1-1 to 1-10 and Comparative Examples 1-1 to 1-4 that a monomer composition having a high polymerization conversion rate upon polymerization could be obtained by performing an impurity removal step of removing at least sulfur from a monomer composition.

Moreover, the following demonstrates that it was confirmed through Examples 2-1 to 2-9 and Comparative Examples 2-1 and 2-2 that a monomer composition having a high polymerization conversion rate upon polymerization could be obtained by performing an impurity removal step of adding any of various adsorbents to a monomer composition and removing at least sulfur from the monomer composition.

### Example 1-1

### <Impurity Removal Step>

After weighing 28 g of a monomer composition containing 2-vinylnaphthalene as a polycyclic aromatic vinyl compound (powder; sulfur content of 150 ppm and bromine content of 300 ppm based on 2-vinylnaphthalene) into a pressure-resistant glass vessel, the monomer composition was dissolved in 84 g of toluene. Thereafter, 14 g of a 3A molecular sieve as a desiccant and 14 g of activated alumina (NKHD-24HD produced by Sumitomo Chemical Co., Ltd.; Al₂O₃: 99.7 mass %; Fe₂O₃: 0.02 mass %; particle diameter: 3 mm; BET specific surface area: 300 m²/g; packing density: 0.77 kg/L) were added, and then the pressure-resistant glass vessel was left at rest at 25° C. for 7 days. Note that the 3A molecular sieve was a desiccant that was added with the aim of adsorption and removal of water molecules contained in the monomer composition and did not contribute to sulfur and halogen removal.

### <Polymerization Step>

A pressure-resistant reactor that had been dried in a nitrogen atmosphere and had been subjected to internal atmosphere purging with nitrogen gas was charged with 30 mL of toluene as a solvent and 32 μL of a 1.6 M hexane solution of n-butyllithium (52 μmol of n-butyllithium) as an anionic polymerization catalyst. Thereafter, 8 g of a 25 mass % toluene solution of 2-vinylnaphthalene (250 equivalents relative to n-butyllithium) that was a purified monomer composition obtained in the above-described “Impurity removal step” that had undergone 7 days of purification treatment was added into the pressure-resistant reactor. A reaction was carried out at 25° C. for 1 hour, a portion of the resultant homopolymer composed of 2-vinylnaphthalene units was subsequently sampled, and various measurements and evaluations were performed as previously described. The results are shown in Table 1.

### Examples 1-2 and 1-3

Various operations, measurements, and evaluations were performed in the same way as in Example 1-1 with the exception that the treatment period in the “Impurity removal step” was changed as shown in Table 1. The results are shown in Table 1.

### Example 1-4

Various operations, measurements, and evaluations were performed in the same way as in Example 1-1 with the exception that the treatment period in the “Impurity removal step” was set as 21 days and the amount of the 25 mass % toluene solution of 2-vinylnaphthalene that was a purified monomer composition added in the “Polymerization step” was changed to 16 g (500 equivalents relative to n-butyllithium). The results are shown in Table 1.

### Example 1-5

A mixed solution of 4 g of a 25 mass % toluene solution of 2-vinylnaphthalene (125 equivalents relative to butyllithium) obtained through the same “Impurity removal step” as in Examples 1-3 and 1-4 that had undergone 21 days of purification treatment and 4 g of a 25 wt % toluene solution of 1-vinylnaphthalene (125 equivalents relative to butyllithium) that had been purified through treatment in the same manner as in purification of the monomer composition containing 2-vinylnaphthalene in Example 1-1 was used as a purified monomer composition. With the exception of this point, various operations, measurements, and evaluations were performed in the same way as in Example 1-1. The results are shown in Table 1.

### <Impurity Removal Step for 1-Vinylnaphthalene>

With the exception that a monomer composition containing 1-vinylnaphthalene as a polycyclic aromatic vinyl compound (liquid; sulfur content of 70 ppm and bromine content of less than 10 ppm based on 1-vinylnaphthalene) was used and that the treatment period was set as 21 days, the monomer composition containing 1-vinylnaphthalene was purified in the same way as in Example 1-1.

### Example 1-6

Various operations, measurements, and evaluations were performed in the same way as in Example 1-1 with the exception that the treatment period in the “Impurity removal step” was set as 28 days and the amount of the 25 mass % toluene solution of 2-vinylnaphthalene that was a purified monomer composition added in the “Polymerization step” was changed to 48 g (1,500 equivalents relative to n-butyllithium). The results are shown in Table 1.

### Example 1-7

The treatment period in the “Impurity removal step” was set as 21 days, and 2 g of isoprene (567 equivalents relative to n-butyllithium), which is an aliphatic conjugated diene compound, was also added in addition to the 25 mass % toluene solution of 2-vinylnaphthalene that was a purified monomer composition in the “Polymerization step”. Moreover, the reaction temperature in the “Polymerization step” was changed to 50° C. In this manner, a random copolymer including 2-vinylnaphthalene units and isoprene units was obtained in the present example. With the exception of these points, various operations, measurements, and evaluations were performed in the same way as in Example 1-1. The results are shown in Table 1.

### Comparative Example 1-1

Various operations, measurements, and evaluations were performed in the same way as in Example 1-1 with the exception that the “Impurity removal step” was not performed, a pretreated monomer composition (25 mass % toluene solution of 2-vinylnaphthalene) was obtained by the following pretreatment, and the pretreated monomer composition was used instead of the purified monomer composition in the “Polymerization step”. The results are shown in Table 1.

### <Pretreatment Step>

The same operations as in the “Impurity removal step” of Example 1-1 were performed with the exception that activated alumina was not used. In other words, a pretreated monomer composition was obtained in this pretreatment step by adding a 3A molecular sieve and performing 7 days of resting so as to remove water from the monomer composition and dry the monomer composition.

### Comparative Example 1-2

In the “Polymerization step”, 8 g of a pretreated monomer composition (25 wt % toluene solution of 2-vinylnaphthalene) obtained in the same way as in Comparative Example 1-1 and 2 g of isoprene (567 equivalents relative to n-butyllithium), which is an aliphatic conjugated diene compound, were added instead of the purified monomer composition. Moreover, the reaction temperature in the “Polymerization step” was changed to 50° C. In this manner, a random copolymer including 2-vinylnaphthalene units and isoprene units was obtained in the present comparative example. With the exception of these points, various operations, measurements, and evaluations were performed in the same way as in Example 1-1. The results are shown in Table 1.

Note that “VN” indicates vinylnaphthalene and “IP” indicates isoprene in the table.

TABLE 1

| | | | Examples | | | | | | | Comparative examples | |
|---|---|---|---|---|---|---|---|---|---|---|---|
| | | | 1-1 | 1-2 | 1-3 | 1-4 | 1-5 | 1-6 | 1-7 | 1-1 | 1-2 |
| Materials | | Polycyclic | 2-VN | 2-VN | 2-VN | 2-VN | 1-VN/ | 2-VN | 2-VN | 2-VN | 2-VN |
| | | aromatic | | | | | 2-VN | | | | |
| | | vinyl compound | | | | | (1:1) | | | | |
| | | Aliphatic conjugated | — | — | — | — | — | — | IP | — | IP |
| | | diene compound | | | | | | | | | |
| Impurity | Sulfur | Treatment period | 7 | 14 | 21 | 21 | 21 | 28 | 21 | 0 | 0 |
| removal | removal | (days) | | | | | | | | | |
| step | | Sulfur content (ppm; | 130 | 109 | 92 | 92 | 62 | 88 | 92 | 150 | 150 |
| | | based on mass of | | | | | | | | | |
| | | polycyclic aromatic | | | | | | | | | |
| | | vinyl compound) | | | | | | | | | |
| | | Ratio relative to pre- | 87 | 73 | 61 | 61 | 61 | 59 | 61 | 100 | 100 |
| | | purification sulfur | | | | | | | | | |
| | | content (mass %) | | | | | | | | | |
| | Halogen | Treatment period | 7 | 14 | 21 | 21 | 21 | 28 | 21 | 0 | 0 |
| | removal | (days) | | | | | | | | | |
| | | Bromine content | 250 | 180 | 150 | 150 | 80 | 143 | 150 | 300 | 300 |
| | | (ppm; based on | | | | | | | | | |
| | | mass of | | | | | | | | | |
| | | polycyclic aromatic | | | | | | | | | |
| | | vinyl compound) | | | | | | | | | |
| | | Ratio relative to pre- | 83 | 60 | 50 | 50 | 50 | 48 | 50 | 100 | 100 |
| | | purification bromine | | | | | | | | | |
| | | content (mass %) | | | | | | | | | |
| Polymerization | | Anionic | n-BuLi | n-BuLi | n-BuLi | n-BuLi | n-BuLi | n-BuLi | n-BuLi | n-BuLi | n-BuLi |
| step | | polymerization | | | | | | | | | |
| | | catalyst | | | | | | | | | |
| | | Introduced amounts | 250 | 250 | 250 | 500 | 250 | 1500 | 250 | 250 | 250 |
| | | of monomers (molar | | | | | | | (VN) + | | (VN) + |
| | | equivalents; based | | | | | | | 567 (IP) | | 567 (IP) |
| | | on anionic | | | | | | | | | |
| | | polymerization | | | | | | | | | |
| | | catalyst) | | | | | | | | | |
| | | Polymerization | 25 | 25 | 25 | 25 | 25 | 25 | 50 | 25 | 50 |
| | | temperature (° C.) | | | | | | | | | |
| Polymer | | Type | Homo- | Homo- | Homo- | Homo- | Random | Homo- | Random | Homo- | Random |
| | | | polymer | polymer | polymer | polymer | copolymer | polymer | copolymer | polymer | copolymer |
| | | Mn (−) | 26000 | 33000 | 23000 | 50000 | 25000 | 134000 | 76000 | 27200 | 13500 |
| | | Mw (−) | 28800 | 37200 | 26000 | 57500 | 32200 | 201000 | 100000 | 33700 | 18400 |
| | | Mw/Mn (−) | 1.11 | 1.13 | 1.13 | 1.15 | 1.29 | 1.5 | 1.31 | 1.24 | 1.36 |
| Conversion rate | | 2-Vinylnaphthalene | 80 | 87 | 95 | 96 | 99 | 88 | >99 | 10 | 8 |
| | | (%) | | | | | | | | | |
| | | 1-Vinylnaphthalene | — | — | — | — | >99 | — | — | — | — |
| | | (%) | | | | | | | | | |
| | | Isoprene (%) | — | — | — | — | — | — | >99 | — | 32 |
| | | Evaluation | B | B | A | A | A | B | A | C | C |

It is clear from Table 1 that a purified monomer composition obtained by a purification method including an impurity removal step that included removing sulfur from a monomer composition, such as in Examples 1-1 to 1-7, had a high polymerization conversion rate upon polymerization. It can also be seen that in a case in which an impurity removal step was not performed, such as in Comparative Examples 1-1 and 1-2, the polymerization conversion rate of a monomer composition was low. In particular, it is clear from comparison of Examples 1-1 and 1-2 with Example 1-3 that when the sulfur content in a monomer composition was adjusted to less than 100 ppm through an impurity removal step, a high molecular weight polymer having a molecular weight of 20,000 or more could be obtained with a high polymerization conversion rate.

### Example 1-8

### <Block Copolymer Production Step>

### <<First Polymerization Step>>

A pressure-resistant reactor that had been dried in a nitrogen atmosphere and undergone purging with nitrogen was charged with 20 mL of toluene as an organic solvent and 32 μL of a 1.6 M hexane solution of n-butyllithium (52 μmol of n-butyllithium) as a polymerization catalyst. Thereafter, 8 g of a 25% toluene solution of 2-vinylnaphthalene (250 equivalents relative to n-butyllithium) that was a purified monomer composition obtained through the same “Impurity removal step” as in Example 1-1 was added as a polycyclic aromatic vinyl compound, and a reaction was performed at 25° C. for 1 hour to carry out a first stage polymerization reaction and obtain a polymer. The number-average molecular weight (Mn), weight-average molecular weight (Mw), peak top molecular weight (Mp), and molecular weight distribution (Mw/Mn) for the obtained polymer were measured as previously described, and the conversion rate of 2-vinylnaphthalene was measured and evaluated as previously described. The results are shown in Table 2.

<<Second Polymerization Step>>

Once the first stage polymerization reaction was completed, 2 g of isoprene (567 equivalents relative to n-butyllithium) as a chain conjugated diene compound was added to the reaction mixture in the pressure-resistant reactor, and a reaction was performed for a further 30 minutes at 50° C. to carry out a second stage polymerization reaction. As a result, a diblock copolymer having a [2-vinylnaphthalene block]-[isoprene block] block configuration was obtained in the reaction mixture. The number-average molecular weight (Mn), weight-average molecular weight (Mw), peak top molecular weight (Mp), and molecular weight distribution (Mw/Mn) of the obtained diblock copolymer were measured as previously described. Moreover, the conversion rate of isoprene added in this step was measured and evaluated as previously described. The results are shown in Table 2. Note that it was confirmed that all 2-vinylnaphthalene remaining after the first stage polymerization reaction had been consumed based on the results of ¹H-NMR measurement.

<<Third Polymerization Step>>

Next, 8 g of a 25% toluene solution of 2-vinylnaphthalene (250 equivalents relative to n-butyllithium) that was a purified monomer composition obtained by the same “Impurity removal step” as in Example 1-1 was added to the reaction mixture as an aromatic vinyl compound and 8.7 of dibutyl ether (1 equivalent relative to n-butyllithium) was added as a co-catalyst, and a reaction was performed at 25° C. for 17 hours to carry out a third stage polymerization reaction. Once the polymerization reaction was completed, 50 μL of methanol was added to end the polymerization reaction. As a result, a triblock copolymer having a [2-vinylnaphthalene block]-[isoprene block]-[2-vinylnaphthalene block] block configuration was obtained in the reaction mixture. The number-average molecular weight (Mn), weight-average molecular weight (Mw), peak top molecular weight (Mp), and molecular weight distribution (Mw/Mn) of the obtained triblock copolymer, and the triblock copolymer purity were measured as previously described. Moreover, the conversion rate of 2-vinylnaphthalene added in this step was measured as previously described. Note that it was confirmed that all isoprene remaining after the second stage polymerization reaction had been consumed based on the results of ¹H-NMR measurement.

### Example 1-9

Various operations, measurements, and evaluations were performed in the same way as in Example 1-8 with the exception that 8 g of a 25% toluene solution of 2-vinylnaphthalene (250 equivalents relative to n-butyllithium) that was a purified monomer composition obtained through the same “Impurity removal step” as in Example 1-2 that had undergone 14 days of purification treatment was added as a polycyclic aromatic vinyl compound in the “First polymerization step” and the “Third polymerization step” in production of a block copolymer. The results are shown in Table 2. Note that it was confirmed that all 2-vinylnaphthalene remaining after the first stage polymerization reaction had been consumed by the time that the second polymerization step was completed and that all isoprene remaining after the second stage polymerization reaction had been consumed by the time that the third polymerization step was completed, in the same way as in Example 1-8.

### Example 1-10

Various operations, measurements, and evaluations were performed in the same way as in Example 1-8 with the exception that 8 g of a 25% toluene solution of 2-vinylnaphthalene (250 equivalents relative to n-butyllithium) that was a purified monomer composition obtained through the same “Sulfur removal step and halogen removal step” as in Example 1-3 that had undergone 21 days of purification treatment was added as a polycyclic aromatic vinyl compound in the “First polymerization step” and the “Third polymerization step” in production of a block copolymer. The results are shown in Table 2. Note that it was confirmed that all 2-vinylnaphthalene remaining after the first stage polymerization reaction had been consumed by the time that the second polymerization step was completed and that all isoprene remaining after the second stage polymerization reaction had been consumed by the time that the third polymerization step was completed, in the same way as in Example 1-8.

### Comparative Example 1-3

Various operations were attempted in order in the same way as in Example 1-8 with the exception that 8 g of a 25% toluene solution of 2-vinylnaphthalene (250 equivalents relative to n-butyllithium) that was a pretreated monomer composition obtained through the same “Pretreatment step” as in Comparative Example 1-1 was added as a polycyclic aromatic vinyl compound in the “First polymerization step” in production of a block copolymer, but the conversion rate in the second polymerization step was less than 5%, and the reaction could not be caused to progress any further. The results of measurements and evaluations that were performed as possible are shown in Table 2.

### Comparative Example 1-4

Various operations were attempted in order in the same way as in Example 1-8 with the exception that 8 g of a 25% toluene solution of 2-vinylnaphthalene (250 equivalents relative to n-butyllithium) that was a pretreated monomer composition obtained through the same “Pretreatment step” as in Comparative Example 1-1 performed with respect to a monomer composition containing 2-vinylnaphthalene as a polycyclic aromatic vinyl compound (powder; sulfur content of 500 ppm and bromine content of less than 20 ppm based on 2-vinylnaphthalene) was added as a polycyclic aromatic vinyl compound in the “First polymerization step” in production of a block copolymer, but the conversion rate in the second polymerization step was less than 5%, and the reaction could not be caused to progress any further. The results of measurements and evaluations that were performed as possible are shown in Table 2.

Note that “VN” indicates vinylnaphthalene and “IP” indicates isoprene in the table.

TABLE 2

| Examples | | | Comparative examples | |
|---|---|---|---|---|
| 1-8 | 1-9 | 1-10 | 1-3 | 1-4 |

| Materials | | Polycyclic aromatic vinyl compound | 2-VN | 2-VN | 2-VN | 2-VN | 2-VN |
|---|---|---|---|---|---|---|---|
| | | Aliphatic conjugated diene compound | IP | IP | IP | IP | IP |
| Imptuity | Sulfur removal | Treatment period (days) | 7 | 14 | 21 | 0 | 0 |
| removal | | Sulfur content (ppm; based on mass of | 130 | 109 | 92 | 150 | 500 |
| step | | polycyclic aromatic vinyl compound) | | | | | |
| | | Ratio relative to pre-purification sulfur | 87 | 73 | 61 | 100 | 100 |
| | | content (mass %) | | | | | |
| | Halogen removal | Treatment period (days) | 7 | 14 | 21 | 0 | 0 |
| | | Bromine content (ppm ; based on mass | 250 | 180 | 150 | 300 | <20 |
| | | of polycyclic aromatic vinyl compound) | | | | | |
| | | Ratio relative to pre-purification | 83 | 60 | 50 | 100 | 100 |
| | | bromine content (mass %) | | | | | |

| Polymerization step | Anionic polymerization catalyst | | n-BuLi | n-BuLi | n-BuLi | n-BuLi | n-BuLi |
|---|---|---|---|---|---|---|---|
| | Introduced amounts of monomers | | 2 g-2 g-2 g | 2 g-2 g-2 g | 2 g-2 g-2 g | 2 g-2 g-2 g | 2 g-2 g-2 g |
| | (2-VN)-(IP)-(2-VN) | | | | | | |
| | Polymer type | | Block | Block | Block | Block | Block |
| | | | copolymer | copolymer | copolymer | copolymer | copolymer |
| | First polymerization | Mw (−) | 41900 | 29700 | 26500 | 33700 | 32200 |
| | step (2-VN) | Mp (−) | 40300 | 30200 | 28400 | 34400 | 33900 |
| | | Mw/Mn (−) | 1.18 | 1.11 | 1.13 | 1.24 | 1.29 |
| | | Conversion | 80 | 87 | >99 | 10 | 8 |
| | | rate (%) | | | | | |
| | Second polymerization | Mw (−) | 114300 | 97200 | 71300 | — | — |
| | step (2-VN)-(IP) | Mp (−) | 118300 | 90200 | 74000 | | |
| | | Mw/Mn (−) | 1.31 | 1.18 | 1.11 | | |
| | | Conversion | 87 | 90 | 93 | <5 | <5 |
| | | rate (%) | | | | | |
| | Third polymerization | Mw (−) | 318500 | 209700 | 136400 | — | — |
| | step (2-VN)-(IP)-(2-VN) | Mp (−) | 282100 | 136200 | 130000 | | |
| | | Mw/Mn (−) | 1.45 | 1.24 | 1.12 | — | — |
| | | Conversion | 80 | 89 | >99 | — | — |
| | | rate (%) | | | | | |

| | | | | | | |
|---|---|---|---|---|---|---|
| Polymer | Purity of triblock copolymer (%) | 49 | 78 | 92 | — | — |

It can be seen from Table 2 that it was possible to achieve a high polymerization conversion rate in a case in which a triblock copolymer having a (2-VN)-(IP)-(2-VN) (i.e., an [A]-[B]-[A]) triblock structure was formed using a purified monomer composition that had been obtained by a purification method including an impurity removal step of removing at least sulfur from a monomer composition in Examples 1-8 to 1-10. On the other hand, it can be seen that in a case in which an impurity removal step was not performed, such as in Comparative Examples 1-3 and 1-4, the polymerization conversion rate of a monomer composition was low. It can also be seen that it was possible to achieve a higher triblock copolymer purity by altering conditions of the impurity removal step, etc., in Examples 1-9 and 1-10.

### Example 2-1

### <Impurity Removal Step>

After weighing 28 g of a monomer composition containing 2-vinylnaphthalene (powder; sulfur content of 150 ppm and bromine content of 300 ppm based on 2-vinylnaphthalene) as a polycyclic aromatic vinyl compound into a pressure-resistant glass vessel, the monomer composition was dissolved in 84 g of toluene. Thereafter, 14 g of a 3A molecular sieve (produced by Tomoe Engineering Co., Ltd.; Al₂O₃ proportional content: less than 40 mass %; cylindrical shape having a diameter of 1.6 mm and a height of 3.2 mm) as a desiccant and 14 g (0.5 (times) based on mass of polycyclic aromatic vinyl compound) of activated alumina (KHO-46 produced by Sumitomo Chemical Co., Ltd.; Fe₂O₃: 0.02 mass %; packing density: 0.80 kg/L) as an adsorbent were added, and then the pressure-resistant glass vessel was left at rest at 25° C. for 21 days. Note that the 3A molecular sieve was a desiccant that was added with the aim of adsorption and removal of water molecules contained in the monomer composition and did not contribute to sulfur and halogen removal.

### <Polymerization Step>

A pressure-resistant reactor that had been dried in a nitrogen atmosphere and had been subjected to internal atmosphere purging with nitrogen gas was charged with 30 mL of toluene as a solvent and 32 μL of a 1.6 M hexane solution of n-butyllithium (52 μmol of n-butyllithium) as an anionic polymerization catalyst. Thereafter, 8 g of a 25 mass % toluene solution of 2-vinylnaphthalene (250 equivalents relative to n-butyllithium) that was a purified monomer composition obtained in the above-described “Impurity removal step” that had undergone 21 days of purification treatment was added into the pressure-resistant reactor. A reaction was carried out at 25° C. for 1 hour, a portion of the resultant homopolymer composed of 2-vinylnaphthalene units was subsequently sampled, and various measurements were performed as previously described. The results are shown in Table 3.

### Example 2-2

Various operations and measurements were performed in the same way as in Example 2-1 with the exception that the additive amount of the adsorbent in the “Impurity removal step” was changed to 28 g, and the additive amount of the adsorbent was set as 1.00 (times) based on the mass of the polycyclic aromatic vinyl compound. The results are shown in Table 3.

### Example 2-3

Various operations and measurements were performed in the same way as in Example 2-1 with the exception that the additive amount of the adsorbent in the “Impurity removal step” was changed to 7 g, and the additive amount of the adsorbent was set as 0.25 (times) based on the mass of the polycyclic aromatic vinyl compound. The results are shown in Table 3.

### Example 2-4

Various operations and measurements were performed in the same way as in Example 2-1 with the exception that 14 g of activated alumina (KHO-24 produced by Sumitomo Chemical Co., Ltd.; Fe₂O₃: 0.02 mass %; packing density: 0.83 kg/L) having a different particle diameter to the adsorbent used in Example 2-1 (smaller particle diameter than in Example 2-1) was added as an adsorbent in the “Impurity removal step”. The results are shown in Table 3.

### Example 2-5

Various operations and measurements were performed in the same way as in Example 2-1 with the exception that 14 g of activated alumina (NKHD-46HD produced by Sumitomo Chemical Co., Ltd.; Fe₂O₃: 0.02 mass %; packing density: 0.74 kg/L) having a different specific surface area to the adsorbent used in Example 2-1 (larger specific surface area than in Example 2-1) was added as an adsorbent in the “Impurity removal step”. The results are shown in Table 3.

### Example 2-6

Various operations and measurements were performed in the same way as in Example 2-1 with the exception that 14 g of activated alumina (NKHD-24HD produced by Sumitomo Chemical Co., Ltd.; Fe₂O₃: 0.02 mass %; packing density: 0.77 kg/L) having a different particle diameter and specific surface area to the adsorbent used in Example 2-1 (smaller particle diameter and larger specific surface area than in Example 2-1) was added as an adsorbent in the “Impurity removal step”, and the reaction time in the “Polymerization step” was changed such that the polymerization conversion rate of 2-vinylnaphthalene was as shown in Table 3. The results are shown in Table 3.

### Example 2-7

A purified monomer composition obtained by adding 14 g of activated alumina (NKHD-24HD produced by Sumitomo Chemical Co., Ltd.) having a different particle diameter and specific surface area to the adsorbent used in Example 2-1 (smaller particle diameter and larger specific surface area than in Example 2-1) as an adsorbent and then performing 7 days of resting at 25° C. was used. In other words, in this example, various measurements were performed with respect to various polymers obtained under the same conditions as in Example 1-8. The results are shown in Table 3.

### Example 2-8

In production of a block copolymer, 8 g of a 25% toluene solution of 2-vinylnaphthalene (250 equivalents relative to n-butyllithium) that was a purified monomer composition obtained by the same “Impurity removal step” as in Example 2-7 with the exception that the purification treatment period was set as 14 days was added as a polycyclic aromatic vinyl compound in the “First polymerization step” and the “Third polymerization step”. In other words, in this example, various measurements were performed with respect to various polymers obtained under the same conditions as in Example 1-9. The results are shown in Table 3.

### Example 2-9

In production of a block copolymer, 8 g of a 25% toluene solution of 2-vinylnaphthalene (250 equivalents relative to n-butyllithium) that was a purified monomer composition obtained by the same “Impurity removal step” as in Example 2-7 with the exception that the purification treatment period was set as 21 days was added as a polycyclic aromatic vinyl compound in the “First polymerization step” and the “Third polymerization step”. In other words, in this example, various measurements were performed with respect to various polymers obtained under the same conditions as in Example 1-10. The results are shown in Table 3.

### Comparative Example 2-1

Various measurements were performed in the same way as in Example 2-1 with the exception that the “Impurity removal step” was not performed, a pretreated monomer composition (25 mass % toluene solution of 2-vinylnaphthalene) was obtained by the following pretreatment, and the pretreated monomer composition was used instead of the purified monomer composition in the “Polymerization step”. The results are shown in Table 3.

### <Pretreatment Step>

The same operations as in the “Impurity removal step” of Example 2-1 were performed with the exception that activated alumina was not used and the treatment period was set as 1 day. In other words, a pretreated monomer composition was obtained in this pretreatment step by adding a 3A molecular sieve and performing 1 day of resting so as to remove water from the monomer composition and dry the monomer composition.

### Comparative Example 2-2

Various operations were attempted in order in the same way as in Example 2-7 with the exception that 8 g of a 25% toluene solution of 2-vinylnaphthalene (250 equivalents relative to n-butyllithium) that was a pretreated monomer composition obtained through the same “Pretreatment step” as in Comparative Example 2-1 was added as a polycyclic aromatic vinyl compound in the “First polymerization step” in production of a block copolymer, but the conversion rate in the second polymerization step was less than 5%, and the reaction could not be caused to progress any further. The results of measurements that were performed as possible are shown in Table 3. Note that it was confirmed that 90% of 2-vinylnaphthalene remaining after the first stage polymerization reaction remained based on the results of ¹H-NMR measurement.

Note that “VN” indicates vinylnaphthalene and “IP” indicates isoprene in the table.

TABLE 3

| | | | Examples | | | | | | | | | Comparative examples | |
|---|---|---|---|---|---|---|---|---|---|---|---|---|---|
| | | | 2-1 | 2-2 | 2-3 | 2-4 | 2-5 | 2-6 | 2-7 (1-8) | 2-8 (1-9) | 2-9 (1-10) | 2-1 | 2-2 |
| Materials | Polycyclic aromatic vinyl compound | | 2-VN | 2-VN | 2-VN | 2-VN | 2-VN | 2-VN | 2-VN | 2-VN | 2-VN | 2-VN | 2-VN |
| | Aliphatic conjugated diene compound | | — | — | — | — | — | — | IP | IP | IP | — | IP |
| Adsorbent | Type | | Activated | Activated | Activated | Activated | Activated | Activated | Activated | Activated | Activated | — | — |
| | | | alumina | alumina | alumina | alumina | alumina | alumina | alumina | alumina | alumina | | |
| | Product number | | KHO-46 | KHO-46 | KHO-46 | KHO-24 | NKHD-46HD | NKHD-24HD | NKHD-24HD | NKHD-24HD | NKHD-24HD | — | — |
| | Al₂O₃ (mass %) | | 99.7 | 99.7 | 99.7 | 99.7 | 99.7 | 99.7 | 99.7 | 99.7 | 99.7 | — | — |
| | Particle diameter (mm) | | 5 | 5 | 5 | 3 | 5 | 3 | 3 | 3 | 3 | — | — |
| | Specific surface area (m²/g) | | 140 | 140 | 140 | 140 | 300 | 300 | 300 | 300 | 300 | — | — |
| | Additive amount (times) | | 0.50 | 1.00 | 0.25 | 0.50 | 0.50 | 0.50 | 0.50 | 0.50 | 0.50 | — | — |
| | (based on polycyclic aromatic) | | | | | | | | | | | | |
| | vinyl compound | | | | | | | | | | | | |
| | Purification treatment period (days) | | 21 | 21 | 21 | 21 | 21 | 21 | 7 | 14 | 21 | — | — |
| Impurity | Sulfur content (ppm) | | 112 | 110 | 125 | 106 | 99 | 92 | 130 | 109 | 92 | 150 | 150 |
| removal step | (based on polycyclic aromatic ) | | | | | | | | | | | | |
| | vinyl compound | | | | | | | | | | | | |
| | Ratio relative to pre-purification | | 75 | 73 | 83 | 71 | 66 | 61 | 87 | 73 | 61 | 100 | 100 |
| | sulfur content (mass %) | | | | | | | | | | | | |
| | Bromine content (ppm) | | 188 | 190 | 202 | 184 | 148 | 150 | 250 | 180 | 150 | 300 | 300 |
| | (based on polycyclic | | | | | | | | | | | | |
| | aromatic vinyl compound) | | | | | | | | | | | | |
| | Ratio relative to pre- | | 63 | 63 | 67 | 61 | 49 | 50 | 83 | 60 | 50 | 100 | 100 |
| | purification bromine content (mass %) | | | | | | | | | | | | |
| Polymerization | Polymer type | | Homo- | Homo- | Homo- | Homo- | Homo- | Homo- | Block | Block | Block | Homo- | Block |
| step | | | polymer | polymer | polymer | polymer | polymer | polymer | copolymer | copolymer | copolymer | polymer | copolymer |
| | Introduced amount of 2-VN monomer (g) | | 2 | 2 | 2 | 2 | 2 | 2 | — | — | — | 2 | — |
| | Introduced amounts of monomers | | — | — | — | — | — | — | 2 g-2 g-2 g | 2 g-2 g-2 g | 2 g-2 g-2 g | — | 2 g-2 g-2 g |
| | (2-VN)-(IP)-(2-VN) | | | | | | | | | | | | |
| | First | Mw (−) | 33,400 | 33,900 | 35,000 | 33,400 | 29,700 | 30,200 | 41,900 | 29,700 | 26,500 | 33,700 | 33,800 |
| | polymerization | MP (−) | 34,500 | 35,000 | 36,800 | 34,700 | 31,400 | 31,500 | 40,300 | 30,200 | 28,400 | 32,400 | 32,100 |
| | step | Mw/Mn (−) | 1.18 | 1.2 | 1.22 | 1.2 | 1.21 | 1.23 | 1.18 | 1.11 | 1.13 | 1.24 | 1.22 |
| | (2-VN) | Conversionrate (%) | 87 | 90 | 85 | 90 | 97 | 99 | 80 | 87 | >99 | 10 | 10 |
| | Second | Mw (−) | — | — | — | — | — | — | 114,300 | 97,200 | 71,300 | — | — |
| | polymerization | MP (−) | — | — | — | — | — | — | 118,300 | 90,200 | 74,000 | — | — |
| | step | Mw/Mn (−) | — | — | — | — | — | — | 1.31 | 1.18 | 1.11 | — | — |
| | (2-VN)-(IP) | Conversion rate (%) | — | — | — | — | — | — | 87 | 90 | 93 | — | <5 |
| | Third | Mw (−) | — | — | — | — | — | — | 318,500 | 209,700 | 136,400 | — | — |
| | polymerization | MP (−) | — | — | — | — | — | — | 282,100 | 136,200 | 130,000 | — | — |
| | step | Mw/Mn (−) | — | — | — | — | — | — | 1.45 | 1.24 | 1.12 | — | — |
| | (2-VN)-(IP)-(2-VN) | Conversion rate (%) | — | — | — | — | — | — | 80 | 89 | >99 | — | — |

| | | | | | | | | | | | | |
|---|---|---|---|---|---|---|---|---|---|---|---|---|
| Polymer | Purity of triblock copolymer (%) | — | — | — | — | — | — | 49 | 78 | 92 | — | — |

It is clear from Table 3 that a purified monomer composition obtained by a purification method including an impurity removal step in which an adsorbent was added to a monomer composition and at least sulfur was removed from the monomer composition, such as in Examples 2-1 to 2-9, had a high polymerization conversion rate upon polymerization. It can also be seen that in a case in which an impurity removal step using an adsorbent was not performed, such as in Comparative Examples 2-1 and 2-2, the polymerization conversion rate of a monomer composition was low.

### INDUSTRIAL APPLICABILITY

According to the present disclosure, it is possible to provide a method of purifying a monomer composition that can at least partially remove impurities capable of impairing a polymerization reaction that are contained in a monomer composition containing a polycyclic aromatic vinyl compound.

Moreover, according to the present disclosure, it is possible to provide a method of producing a polymer using a composition that contains at least a polycyclic aromatic vinyl compound-containing monomer composition that has been purified.

1. A method of purifying a monomer composition containing a polycyclic aromatic vinyl compound including at least two monocycles selected from the group consisting of aromatic hydrocarbon monocycles and aromatic heteromonocycles, comprising
an impurity removal step including removing at least sulfur from the monomer composition.

2. The method of purifying a monomer composition according to claim 1, wherein sulfur content in the monomer composition is adjusted to 150 ppm or less based on mass of the polycyclic aromatic vinyl compound in the impurity removal step.

3. The method of purifying a monomer composition according to claim 1, wherein sulfur content in a desulfurized monomer composition obtained through the impurity removal step is 90 mass % or less of sulfur content in the monomer composition prior to purification.

4. The method of purifying a monomer composition according to claim 1, further comprising removing halogen from the monomer composition in the impurity removal step.

5. The method of purifying a monomer composition according to claim 4, wherein halogen content in the monomer composition is adjusted to 300 ppm or less based on mass of the polycyclic aromatic vinyl compound in the impurity removal step.

6. The method of purifying a monomer composition according to claim 4, wherein halogen content in a dehalogenated monomer composition obtained through the impurity removal step is 90 mass % or less of halogen content in the monomer composition prior to purification.

7. The method of purifying a monomer composition according to claim 1, wherein the polycyclic aromatic vinyl compound includes vinylnaphthalene.

8. The method of purifying a monomer composition according to claim 1, wherein sulfur is removed from the monomer composition by adding an adsorbent to the monomer composition in the impurity removal step.

9. The method of purifying a monomer composition according to claim 8, wherein the adsorbent includes more than 50 mass % of Al₂O₃.

10. The method of purifying a monomer composition according to claim 8, wherein the adsorbent has a BET specific surface area of 100 m²/g or more.

11. The method of purifying a monomer composition according to claim 8, wherein the adsorbent is added in a proportion of at least 0.05 times mass of the polycyclic aromatic vinyl compound in the impurity removal step.

12. A method of producing a polymer comprising a polymerization step of performing anionic polymerization of a composition (I) containing a purified monomer composition obtained by the method of purifying a monomer composition according to claim 1 to obtain a polymer.

13. The method of producing a polymer according to claim 12, further comprising copolymerizing the polycyclic aromatic vinyl compound with an aliphatic conjugated diene compound in the polymerization step.

14. The method of producing a polymer according to claim 13, further comprising block copolymerizing the polycyclic aromatic vinyl compound and the aliphatic conjugated diene compound to obtain a block copolymer in the polymerization step.

15. The method of producing a polymer according to claim 13, further comprising randomly copolymerizing the polycyclic aromatic vinyl compound and the aliphatic conjugated diene compound to obtain a random copolymer in the polymerization step.
